(19) United States
(12) Patent Application Publication
(10) Pub. No.: US 2003/0083210 A1
(43) Pub. Date: May 1, 2003

(54) Lamellar post foaming cleansing composition and dispensing system

(75) Inventors: Jessica Weiss Goldberg, Fairfield, CT (US); Christine E. Hayward, Torrington, CT (US); Virgilio B. Villa, Emerson, NJ (US); William F. Furrier, Sandy Hook, CT (US)

Correspondence Address: UNILEVER, PATENT DEPARTMENT, 45 RIVER ROAD, EDGEWATER, NJ, 07020, US

(73) Assignee: Unilever Home and Personal Care USA, Division of CONOPCO, INC.

(21) Appl. No.: 09/938,455

(22) Filed: Aug. 24, 2001

(51) Int. Cl. A61K 7/50

(52) U.S. Cl. 510/130; 510/156; 510/424

(57) ABSTRACT

An aqueous self-foaming liquid cleansing composition comprising a base lotion composition having a surfactant system containing an anionic surfactant, and a post-foaming agent, wherein the composition has a shear-thinning lamellar structure is described. In another embodiment, a low cost dispensing system is described for use with the inventive composition. In a futher embodiment, the inventive composition is substantially free of soap.

### BACKGROUND OF THE INVENTION

1. Field of the Invention

The present invention relates to detergent compositions suitable for topical application for cleansing the human body, such as the skin and hair. In particular, it relates to self-foaming lamellar compositions, and to aerosol barrier dispensing systems to dispense the self-foaming lamellar compositions.

2. The Related Art

The following all disclose post-foaming isotropic gels: PCT publication no.WO 9703646 to C. Hall, et al., Published Feb. 6, 1997 discloses a post-foaming isotropic gel with a base material consisting of a detergent and a thickener, of viscosity of at least 9,500 cps; U.S. Pat. No. 4,772,427 to A. Dawson, et al. issued Sep. 20, 1988, discloses a post-foaming isotropic gel shower product with a viscosity of between 20,000-100,000 cps; EPO publication no. EP 0987018 to T. McGee et al., published Mar. 22, 2000, discloses a post-foaming isotropic shower gel with a viscosity of between 1000-60,000 cps; U.S. Pat. No. 5,248,495 to T. Patterson, et al., issued Sep. 28, 1993, discloses a post foaming shaving gel composition having a viscosity of 15-16,000 cps.

The following all disclose mousses or moisturizing personal cleansing compositions with aerosol propellants: PCT publication no. WO9938491 to T. Dixon, et al. published Aug. 5, 1999 discloses moisturizing personal cleansing compositions with improved lipid deposition with a viscosity of 300-100,000 cps and a skin lipid deposition of greater than 25 mg/cm² on the skin; PCT publication no. WO9827936 to G. Dawson, et al., Published Jul. 2, 1998, discloses a packaged personal cleansing product with a viscosity less than 700 mPa.s using a Brookfield #2 spindle at 60 rpm and NO or CO₂ or mixtures thereof as foaming agents; U.S. Pat. No. 5,002,680 to R. Schmidt, et al. issued Mar. 26, 1991 discloses a mild skin cleansing aerosol nonsoap mousse with skin feel and moisturization benefits; PCT publication no.WO9938490 to T. Dixon, Published Aug. 5, 1999 dicloses an aerosol personal cleansing emulsion compositions which contain low vapor pressure propellants.

U.S. Pat. No. 5,186,857 to M. VISHNUPAD et al., issued Feb. 16, 1993, discloses a self-foaming high oil containing, non-lamellar composition of at least 10% having at least 5% of one high foaming surface active agent which increases the solubility of said foam-producing liquid in said oil or oil/wax mixture; one or more foam suppressing agents which cooperate with said high foaming surface active agents to help solubilize the hydrocarbon in the oil mixture, and a process for making and using same,

U.S. Pat. No. 5,091,111 to P. Neumiller, issued Feb. 25, 1992

discloses a vesicular structured aqueous emulsion containing nonionic surfactants and an aerosol delivery system. However the composition taught contains no anionic surfactants or emollients.

The above patents and publications however, fail to disclose or suggest a post-foaming cleansing composition based on a lamellar structured fluid that is shear thinning and provides excellent stability for high concentrations of lipophillic emollients, greater moisturization and sensory benefits, and greater and more consistent dispensing control than prior art post-foaming cleansing compositions. Furthermore, the above patents and publications fail to teach a cost effective dispensing method to dispense the inventive composition using an aerosol barrier can, preferably a piston type can. The inventive cleansing composition can find application for body washes, shaving creams, hair mousses, shampoos, conditioners, scalp treatments, hand or body lotion, and facial creams. Post foaming is herein defined as a material which remains substantially free from foaming for a short time after dispensing from a pressurized can and eventually forms a higher density product as compared to a mousse which is dispensed immediately as a foam and forms a lower density product. Prior art mousse compositions are disadvantageous because they tend to break down and dissipate quickly after being applied to the skin. Post-foaming gels are generally packaged in aerosol barrier containers which separate the propellant from the blended cleansing and foaming agent product. Mousses are packaged with the propellant blended with the cleansing product.

### SUMMARY OF THE INVENTION

In one aspect the present invention comprises a liquid cleansing and moisturizing composition comprising: (a) from about 80 to about 97% by wt. of a neat cleansing lotion having about 0.5 to about 65% by wt. of the total composition of at least one non-soap anionic or mixture of non-soap anionic surfactants; about 35 to about 90% by wt. of the total composition of water;

(b) from about 3 to about 20% by wt. of the total composition of at least one volatile foaming agent, and wherein the neat cleansing lotion is a lamellar structured shear thinning composition at 25 C.

In order to convey a heaping lotion appearance and excellent sensory characteristics, the inventive neat cleansing lotion preferably has a shear thinning index greater than about 0.4, more preferably greater than about 0.5. Shear thinning index is defined below.

In another embodiment, a liquid cleansing and moisturizing composition and dispensing system is provided comprising: (a) from about 80 to about 97% by wt. of a neat cleansing lotion having about 0.5 to about 65% by wt. of the total composition of at least one non-soap anionic or mixture of non-soap anionic surfactants; about 35 to about 90% by wt. of the total composition of water; (b) from about 3 to about 20% by wt. of the total composition of a volatile foaming agent; wherein the neat cleansing lotion is a lamellar structured shear thinning composition at 25 C; and wherein the cleansing composition is contained in an aerosol pressurized container having a barrier separating the cleansing composition from an aerosol propellant preferably a piston type can.

In another embodiment, the inventive liquid cleansing and moisturizing composition and dispensing system is substantially soap free and comprises: a) from about 80 to about 97% by wt. of a neat cleansing lotion having about 0.5 to about 65% by wt. of the total composition of at least one non-soap anionic or mixture of non-soap anionic surfactants; less than about 4%, preferably less than about 1%, and most preferably less than about 0.5% of a soap; about 35 to about 90% by wt. of the total composition of water; and (b) from about 3 to about 20% by wt. of the total composition of a volatile foaming agent; and wherein the neat cleansing lotion is a lamellar structured shear thinning composition at 25 C; and wherein the cleansing composition is contained in an aerosol pressurized piston container having an aerosol propellant, preferably a piston type can.

### BRIEF DESCRIPTION OF THE DRAWINGS

The foregoing features, advantages, and objects of this invention are now described in more detail with reference to the drawings in which:

FIG. 1 is a graph of the mean visual scores for the dryness data from the skin moisturization study.

FIG. 2 is a graph of the average replicate scores for the Skicon data from the skin moisturization study.

FIG. 3 is a graph of the average replicate scores for the Corneometer data from the skin moisturization study.

FIG. 4 is a graph of viscosity versus shear stress for comparative commercially available Edge® gel at 4° C.

FIG. 5 is a graph of viscosity versus shear stress for inventive lamellar lotion concentrates EE and GG with and without added heptane compared to comparative isotropic lotion base concentrate A with and without added heptane.

FIG. 6 is an expanded version of FIG. 5 showing in more detail comparative isotropic lotion base concentrate A with and without added heptane.

FIG. 7 shows a side by side comparison of the stability of the foam produced by an inventive lamellar post foaming lotion compared to a comparative isotropic post-foaming gel.

### DETAILED DESCRIPTION OF THE INVENTION

In one aspect the present invention comprises a liquid cleansing and moisturizing composition comprising:

(a) from about 80 to about 97% by wt. of a neat cleansing lotion having

about 0.5 to about 65%, preferably 1 to about 25% by wt. of the total composition of at least one non-soap anionic or mixture of non-soap anionic surfactants;

about 35 to about 90% by wt. of the total composition of water;

(b) from about 3 to about 20% by wt. of the total composition of at least one volatile foaming agent, preferably having at least one or more hydrocarbons or mixture thereof; and

wherein the neat cleansing lotion is a lamellar structured shear thinning composition at 25 C.

Advantageously, the neat cleansing lotion further comprises about 0.1 to about 25% by wt. of the total composition of a surfactant selected from amphoteric, zwitterionic or mixtures thereof. Preferably the neat cleansing lotion further comprises about 0.5 to about 50%, more preferably 6 to about 35 by wt. of the total composition of lipophilic emollients, humectants, and mixtures thereof. Advantageously the lipophilic emollient is selected from a triglyceride oil, mineral oil, petrolatum, and a blend thereof, and the like; and the humectants are selected from polyhydric alcohols, polyols, and blends thereof, and the like.

Advantageously, the neat cleansing lotion contains at least one lipophilic emollient in a concentration greater than about 10%, preferably greater than about 12% by weight of the total composition. Preferably this lipophilic emollient includes at least one triglyceride oil.

Advantageously the inventive neat cleansing lotion contains about 0.1% to about 15% by wt. of the total composition of a lamellar phase inducing structurant selected from: C8 to C24 alkenyl or branched alkyl fatty acid or ester thereof with a melting point below 25C; C8 to C24 alkenyl or branched alkyl fatty alcohol or ether thereof with melting point below 25C; C5 to C12 alkyl fatty acids; and hydroxystearin, and the like. Preferably the lamellar structurant is selected from isostearic acid, lauric acid, oleic acid, palm kernel acid, coconut acid, and blends thereof, and the like.

In order to convey a heaping lotion appearance and excellent sensory characteristics, the inventive neat cleansing lotion has a shear thinning index greater than about 0.4, preferably greater than about 0.5. Shear thinning index is defined below.

Preferably the inventive neat cleansing lotion further comprises at least one cationic skin conditioning agent, preferably present in the range of from about 0.01 to about 5%, more preferably about 0.1 to about 1% by wt. of the total composition. Advantageously the cationic skin conditioning agent is selected from cationic polysaccharides, cationic copolymers of saccharides and synthetic cationic monomers, synthetic cationic polymers, polymeric quaternary ammonium salts of hydroxyethylcellulose, cationic proteins, and salts and derivatives thereof, and the like.

With regards to the surfactants present in the inventive neat cleansing lotion, the anionic surfactant is preferably selected from alkyl ether sulfate, alkyl sulfate, acyl isethionate, mono-and di-alkyl phosphate, and blends thereof, and the like, and the amphoteric/zwitterionic surfactant is preferably selected from cocoamidopropyl betaine, sodium lauroamphoacetate, sodium cocoamphoacetate and blends thereof, and the like.

The combined inventive cleansing lotion and foaming agent has sufficient initial viscosity to convey excellent sensory feel. Preferably the initial viscosity is greater than about 40,000 cps measured at 10 Pa at 25 C according the controlled shear stress rheological method defined below. More preferably the initial viscosity is in the range of about 40,000 to about 2,000,000 cps at 10 Pa and 25° C.

The inventive neat cleansing lotion advantageously comprises a solubilizing agent, preferably selected from isopropyl palmitate and isopropyl myristate, and the like. In addition, the inventive neat cleansing lotion advantageously contains about 0.1% to about 5% by wt. of a lamellar stabilizing material consisting of a polymeric hydrophilic emulsifier modified at one or both ends with hydrophobic polyhydroxy fatty acid ester chain; preferably the emulsifier is dipolyhydroxystearate. In addition, the emulsifier preferably has a polyalkylene glycol backbone chain of general formula: H (0 (CH2) a) nOH wherein a is 2 to 4 and n is 2 to 60 having 1 to 50 C8 to C24 fatty acid group or groups attached to one or both sides of the backbone. More preferably the fatty acid group or groups attached to backbone chain are selected from hydroxystearic acid, palmitic acid, and blends thereof, and the like.

In another embodiment, a liquid cleansing and moisturizing composition and dispensing system is provided comprising: (a) from about 80 to about 97% by wt. of a neat cleansing lotion having about 0.5 to about 65%, preferably about 1 to about 25 by wt. of the total composition of at least one non-soap anionic or mixture of non-soap anionic surfactants; about 35 to about 90% by wt. of the total composition of water; (b) from about 3 to about 20% by wt. of the total composition of a volatile foaming agent, preferably a hydrocarbon foaming agent; wherein the neat cleansing lotion is a lamellar structured shear thinning composition at 25 C; and wherein the cleansing composition is contained in an aerosol pressurized container having a barrier separating the cleansing composition from an aerosol propellant, more preferably an aerosol piston can.

Advantageously, the composition dispensed by the inventive dispensing system has a dynamic density of greater that about 0.2 g/ml, preferably about 0.4 g/ml as measured within 30 seconds after dispensing at 25 C and 1 atm pressure using the methodology provided below.

In another embodiment, the inventive liquid cleansing and moisturizing composition and dispensing system comprises: a) from about 80 to about 97% by wt. of a neat cleansing lotion having about 0.5 to about 65%, preferably about 1 to about 25% by wt. of the total composition of at least one non-soap anionic or mixture of non-soap anionic surfactants; less than about 4%, preferably less than about 1%, and most preferably less than about 0.5% of a soap; about 35 to about 90% by wt. of the total composition of water; and (b) from about 3 to about 20% by wt. of the total composition of a volatile foaming agent preferably containing at least one volatile hydrocarbon; and wherein the neat cleansing lotion is a lamellar structured shear thinning composition at 25 C; and wherein the cleansing composition is contained in an aerosol pressurized piston container having an aerosol propellant, preferably a piston type can.

Lamellar Cleansing Lotion Microstructure

The rheological behavior of all surfactant solutions, including liquid cleansing solutions, is strongly dependent on the microstructure, i.e., the shape and concentration of micelles or other self-assembled structures in solution. When there is sufficient surfactant to form micelles (concentrations above the critical micelle concentration or CMC), for example, spherical, cylindrical (rod-like) or discoidal micelles may form. AS surfactant concentration increases, ordered liquid crystalline phases such as lamellar phase, hexagonal phase or cubic phase may form. The lamellar phase, for example, consists of alternating surfactant bilayers and water layers. These layers are not generally flat but fold to form submicron spherical onion like structures called vesicles or liposomes. The hexagonal phase, on the other hand, consists of long cylindrical micelles arranged in a hexagonal lattice. In general, the microstructure of most personal care products consist of either spherical micelles; rod micelles; or a lamellar dispersion.

As noted above, micelles may be spherical or rod-like. Formulations having spherical micelles tend to have a low viscosity and exhibit newtonian shear behavior (i.e., viscosity stays constant as a function of shear rate; thus, if easy pouring of product is desired, the solution is less viscous and, as a consequence, it doesn't suspend as well). In these systems, the viscosity increases linearly with surfactant concentration.

Rod micellar solutions are more viscous because movement of the longer micelles is restricted. At a critical shear rate, the micelles align and the solution becomes shear thinning. Addition of salts increases the size of the rod micelles thereof increasing zero shear viscosity (i.e., viscosity when sitting in bottle) which helps suspend particles but also increases critical shear rate (point at which product becomes shear thinning; higher critical shear rates means product is more difficult to pour).

Lamellar dispersions differ from both spherical and rod-like micelles because they can have high zero shear viscosity (because of the close packed arrangement of constituent lamellar droplets), yet these solutions are very shear thinning (readily dispense on pouring). That is, the solutions can become thinner than rod micellar solutions at moderate shear rates.

In formulating liquid cleansing compositions, therefore, there is the choice of using rod-micellar solutions (whose zero shear viscosity, e.g., suspending ability, is not very good and/or are not very shear thinning); or lamellar dispersions (with higher zero shear viscosity, e.g. better suspending, and yet are very shear thinning). The use of lamellar dispersions is important for the present invention.

Surfactants:

Surfactants are an essential component of the inventive self-foaming cleansing composition. They are compounds that have hydrophobic and hydrophilic portions that act to reduce the surface tension of the aqueous solutions they are dissolved in. Useful surfactants can include anionic, nonionic, amphoteric, and cationic surfactants, and blends thereof.

Anionic Surfactants:

The self-foaming cleansing composition of the present invention contains one or more anionic detergents. The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:

RO(CH₂CH₂O)ₙSO₃M

wherein R is an alkyl or alkenyl having 8 to 18 carbons, preferably 12 to 18 carbons, n has an average value of greater than 1.0, preferably greater than 3; and M is a

solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:

R⁴O₂CCH₂CH(SO₃M)CO₂M; and

amide-MEA sulfosuccinates of the formula;

R⁴CONHCH₂CH₂O₂CCH₂CH(SO₃M)CO₂M

wherein R⁴ ranges from C₈-C₂₂ alkyl and M is a solubilizing cation.

Sarcosinates are generally indicated by the formula:

R¹CON(CH₃)CH₂CO₂M,

wherein R¹ ranges from C₈-C₂₀ alkyl and M is a solubilizing cation.

Taurates are generally identified by formula:

R²CONR³CH₂CH₂SO₃M

wherein R² ranges from C₈-C₂₀ alkyl, R³ ranges from C₁-C₄ alkyl and M is a solubilizing cation.

The inventive self-foaming cleansing composition contains anionic surfactants, preferably contains C₈-C₁₈ acyl isethionates. These esters are prepared by reaction between alkali metal isethionate with mixed aliphatic tatty acids having from 6 to 18 carbon atoms and an iodine value of less than 20. At least 75% of the mixed fatty acids have from 12 to 18 carbon atoms and up to 25% have from 6 to 10 carbon atoms.

Total surfactants will generally range from about 0.5% to about 65% by weight of the self-foaming cleansing composition. Preferably, this component is present from about 2% to about 50% in the self-foaming cleansing composition.

The acyl isethionate may be an alkoxylated isethionate such as is described in llardi et al., U.S. Pat. No. 5,393,466, titled “Fatty Acid Esters of Polyalkoxylated isethonic acid; issued Feb. 28, 1995; hereby incorporated by reference. This compound has the general formula:

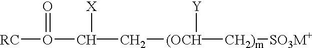

wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Amphoteric Surfactants

One or more amphoteric surfactants may be used in this invention. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

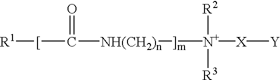

where R¹ is alkyl or alkenyl of 7 to 18 carbon atoms;

R² and R³ are each independently alkyl, hydroxyalkyl or carboxyalkyl of 1 to 3 carbon atoms;

n is 2 to 4;

m is 0 to 1;

X is alkylene of 1 to 3 carbon atoms optionally substituted with hydroxyl, and

Y is —CO₂— or —SO₃—

Suitable amphoteric surfactants within the above general formula include simple betaines of formula:

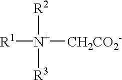

and amido betaines of formula:

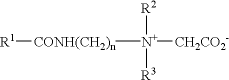

where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula:

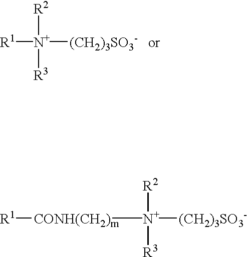

where m is 2 or 3, or variants of these in which —(CH₂)₃ SO₃— is replaced by

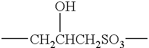

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the self-foaming cleansing composition of the present invention.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

An optional component in compositions according to the invention is a cationic skin feel agent or polymer, such as for example cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR Cl 3S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Cationic Surfactants

One or more cationic surfactants may also be used in the inventive self-foaming cleansing composition.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued March, 27, 1973; and “Surface Active Agents and Detergents” (Vol. I & II) by Schwartz, Perry & Berch, both of which are also incorporated into the subject application by reference.

In addition, the inventive self-foaming cleansing composition composition of the invention may include 0 to 15% by wt. optional ingredients as follows:

perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise antimicrobials such as 2-hydroxy-4,2′, 4′trichlorodiphenylether (DP300); preservatives such as dimethyloldimethylhydantoin (Glydant XL 000), parabens, sorbic acid etc., and the like.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

Humectants such as polyhydric alcohols, e.g. glycerine and propylene glycol, and the like; and polyols such as the polyethylene glycols listed below and the like may be used.

Polyox WSR-205 PEG 14M,

Polyox WSR-N-60K PEG 45M, or

Polyox WSR-N-750 PEG 7M.

The emollient “composition” may be a single benefit agent component or it may be a mixture of two or more compounds one or all of which may have a beneficial aspect. In addition, the benefit agent itself may act as a carrier for other components one may wish to add to the self-foaming cleansing composition composition.

A blend of a hydrophobic and hydrophilic emollients may be used. Preterably, hydrophobic emollients are used in excess of hydrophilic emollients in the inventive self-foaming cleansing composition. Most preferably one or more hydrophobic emollients are used alone. Hydrophobic emollients are preferably present in a concentration greater than about 10% by weight, more preferably about 12% by weight. The term “emollient” is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

Useful emollients include the following:

(a) silicone oils and modifications thereof such as linear and cyclic polydimethylsiloxanes; amino, alkyl, alkylaryl, and aryl silicone oils;

(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;

(c) waxes such as carnauba, spermaceti, beeswax, lanolin, and derivatives thereof;

(d) hydrophobic and hydrophillic plant extracts;

(e) hydrocarbons such as liquid paraffins, vaseline, microcrystalline wax, ceresin, squalene, pristan and mineral oil;

(f) higher fatty acids such as lauric, myristic, palmitic, stearic, behenic, oleic, linoleic, linolenic, lanolic, isostearic, arachidonic and poly unsaturated fatty acids (PUFA);

(g) higher alcohols such as lauryl, cetyl, stearyl, oleyl, behenyl, cholesterol and 2-hexydecanol alcohol;

(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;

(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe Vera, menthol, cineole, eugenol, citral, citronelle, borneol, linalool, geraniol, evening primrose, camphor, thymol, spirantol, penene, limonene and terpenoid oils;

(j) lipids such as cholesterol, ceramides, sucrose esters and pseudo-ceramides as described in European Patent Specification No. 556,957;

(k) vitamins, minerals, and skin nutrients such as milk, vitamins A, E, and K; vitamin alkyl esters, including vitamin C alkyl esters; magnesium, calcium, copper, zinc and other metallic components;

(l) sunscreens such as octyl methoxyl cinnamate (Parsol MCX) and butyl methoxy benzoylmethane (Parsol 1789);

(m) phospholipids;

(n) antiaging compounds such as alpha hydroxy acids, beta hydroxy acids; and

(O) mixtures of any of the foregoing components, and the like.

Preferred emollient benefit agents are selected from triglyceride oils, mineral oils, petrolatum, and mixtures thereof. Further preferred emollients are triglycerides such as sunflower seed oil.

Dispensing systems; Propellants and Post-foaming agents:

Suitable aerosol barrier can dispensing systems include bag in can, pressurized bladder type packaging, piston type packaging, and the like. Especially preferred is the piston type packaging for ease of production and low cost.

Dispensing systems according to the invention have two associated gases; a propellant gas and a foaming agent. The propellant gas is that which is contained within the can, but acts against the outside of the bag, barrier, or piston in which the composition is contained to dispense the composition when an actuator on the can is used. The propellant gas can be any suitable gas, but is conveniently a liquifiable volatile hydrocarbon, such as isobutane or blends thereof, though any propellant which would function to dispense the composition would be suitable. The propellant gas is present in the packaged composition at any required and suitable level, but is typically present in the packaged product at levels sufficient to produce a satisfactory dispensing pressure, which will typically be 29-174 psi, more preferably 43-135 psi.

The other component required in the inventive compositions according the invention is a foaming agent. The foaming agent is present in compositions according to the invention to allow the composition, which is dispensed in the form of a shear thinning lotion, once dispensed to generate a foam, as it evaporates on contact with room temperature air, even more rapidly on contact with a skin surface. The generation of a foam provides a product which has various desirable consumer attributes, including ease of handling and spreading, and desirable sensory properties.

Suitable post foaming agents for inclusion in compositions according to the invention can include any gas or volatile liquid that is soluble or dispersible in the composition. Especially preferred are hydrocarbons, such as isobutane and isopentane. Foaming agents are present in the inventive compositions according to the invention at levels of 3-20%, preferably 4-15%, and more preferably 5-10% by weight of the total composition.

Suitable foaming agents preferably are capable of being contained in compositions according to the invention as liquids, which may have been formed under the pressure to which the packaged composition has been subjected. As such, it is also preferable that packages in which the inventive composition is stored have little to no head space, to prevent the premature evaporation of the post foaming agent.

The composition may also comprise decorative or functional particulates including speckles, coloured or reflective particles, or shaped particles, encapsulated beads, sponge, and the like.

Except in the operating and comparative examples, or where otherwise explicitly indicated, all numbers in this description indicating amounts of material ought to be understood as moditied by the word “about”.

The following examples will more fully illustrate the embodiments of this invention. All parts, percentages and proportions referred to herein and in the appended claims are by weight unless otherwise illustrated. Physical test methods are described below:

### EXAMPLE 1

### Skin Moisturization Clinical Test Results

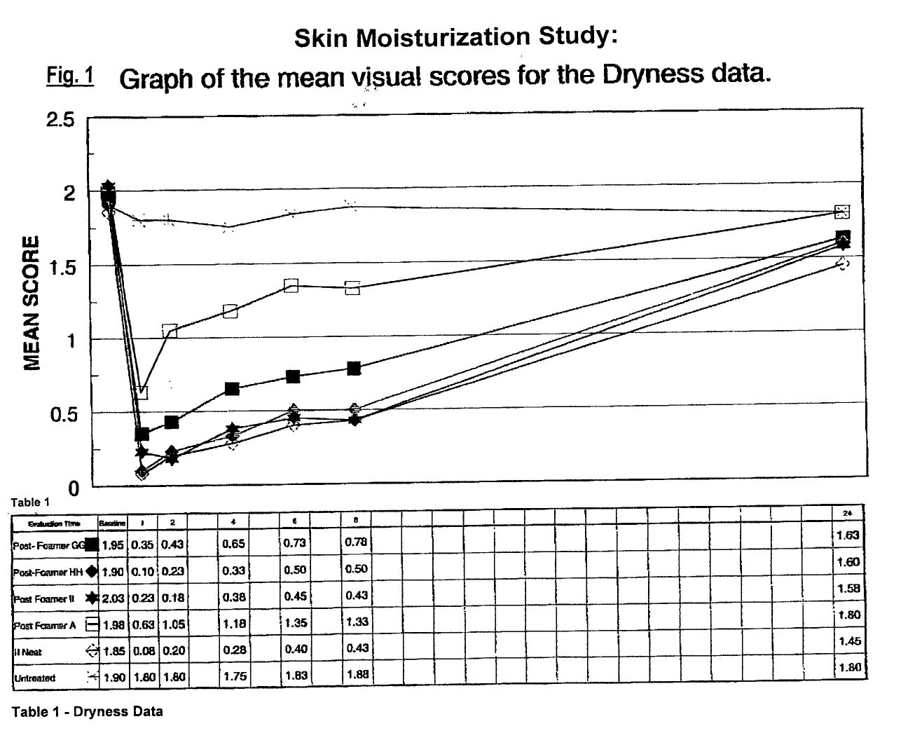

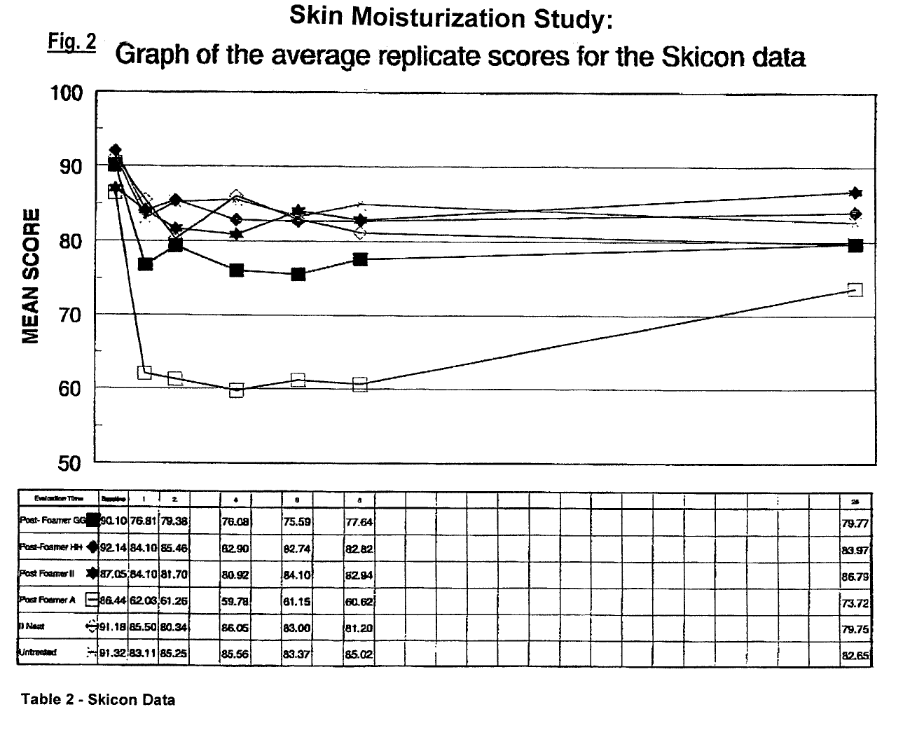

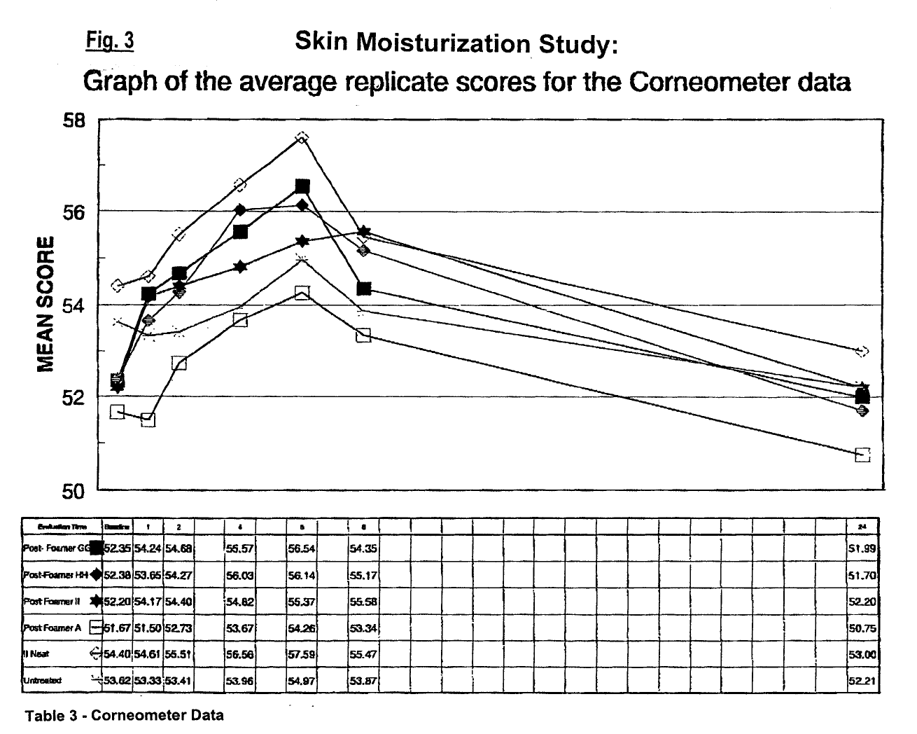

Skin moisturization clinical test results (see methodology below) were evaluated for inventive and comparative formulations described in tables 4 and 5. Processes for preparing the comparative isotropic gel formulations of table are described in Table 7. The skin moisturization clinical test results are displayed in Tables 1-3 respectively; and graphically depicted according to visual evaluation, FIG. 1; Corneometer data, FIG. 2; and Skicon data, FIG. 3,

Comparative sample A with a hydroxypropylmethyltrimonium chloride level of 0.27 wt % and hydroxymethylcellulose at 0.15% filled into the bag-in-can container was tested against Inventive examples GG,HH,II, (also filled into bag-in-cans) and II neat in both the skin moisturization clinical and sensory tests see Example 2).

FIG. 1 (Visual dryness) shows that the most effective (least visual dryness) product(s) are Inventive II Neat, Inventive Post foamer II, and Inventive Post-foamer HH. Inventive Post-foamer GG is slightly less effective (and more visual dryness), but this difference is not statistically significant. However, Comparative Post-foamer A is significantly less effective (with significantly more visual dryness).

Skicon (FIG. 2) and corneometer (FIG. 3) both are measures of skin hydration (and thus moisturization) based on measuring electrical properties using different parameters. The skicon measures skin conductance which emphasizes the hydration at or very close to the skin surface. The data for all samples except comparative post-foamer A are clustered together; the data for comparative post-foamer A shows that using it leads to significantly less hydrated skin compared to the other samples.

Corneometer (FIG. 3) measures capacitance which is primarily a bulk effect which is less sensitive to surface behavior. However, the least moisturizing product (lowest trace on FIG. 3) is that of comparative post-foamer A.

### EXAMPLE 2

### Lather and Sensory Effect

The lather and sensory effect of the inventive composition HH in bag-in-can was compared to an isotropic gel comparative formulation A in bag-in-can using monadic test subject data from a proto-monadic test of 89 subjects with a balanced order of presentation. Each product was evaluated for 1 week. Subjects showered at least four times a week with each sample. It was seen that the inventive product provides a “dense, soft foam” based on the following data:

87% agree completely or somewhat that it has rich creamy lather

78% agree completely or somewhat that has rich luxurious cleansing foam

69% agree completely or somewhat that the product has a pleasing consistency

77% agree completely or somewhat that the product rinses quickly and easily

76% agree completely or somewhat that it doesn't leave a residue

Sensory benefits were evaluated in monadic test having 31 subjects in the inventive cell and 47 in comparative cell. The following results were found (percent agree completely and agree somewhat):

| Sensory benefit | Comparative | Inventive |
|---|---|---|
| Does not dry your skin | 65 | 90 |
| Makes your skin feel soft | 78 | 84 |
| Leaves your skin feeling silky | 61 | 78 |
| Moisturizing your skin | 48 | 81 |
| Leaves your skin feeling smooth | 74 | 87 |
| Makes your skin feel healthy | 45 | 65 |

### EXAMPLE 3

### Aerosol Piston Can Performance

The function of inventive lamellar and comparative isotropic formulations in an aerosol piston can as described in tables 4 and 5 was examined. Processing and can filling directions are listed below.

Two failure modes became apparent when working with the comparative isotropic formulations. For proper functioning, It is critical for the lotion base and foaming agent(s) to mix well and show no separation. If there is separation, then it is possible in production to see variation from can to can in foaming agent content, or have complete separation and have no foaming agent in the can, or conversely have all foaming agent (and little or no product) in the can. Alternatively, with those isotropic samples that did mix well, blow-by was observed in the piston can. Blow-by refers to an insufficient seal of the product+foaming agent mixture at the junction between the piston and the edges of the can. If such a junction is insufficient, the propellant and the product can each “blow-by” the piston, resulting either in propellant above the piston, or product below the piston. Failed comparative cans were dissected after depressurizing by perforating the can on the “propellant side” of the piston. Usually a gel emerged from the perforation demonstrating that blow-by had occurred i.e. a substantial quantity of product was under the piston in place of the propellant.

In all the above formulations, if the base and volatile foaming agents mixed well, the cans dispensed product successfully immediately after filling. However, for all the cases above that did mix, after the stresses due to shipping, product was unable to be dispensed successfully. On depressing the actuator, either no material was dispensed, or a very small quantity was dispensed (total foam; not post-foaming), or propellant gas came out (hissing with no product). Such a failure to dispense may imply that blowby occurred sometime during the time between initial testing and after shipping.

A screening test was developed wherein 6 wt % of heptane as a model for a hydrocarbon foaming agent, was added to 94 wt % of the lotion base concentrate. If the two mixed well, it was considered to be a potential candidate for filling into the piston cans. Although it was observed that our thicker systems did in fact mix well with the hydrocarbons in lab, it was observed that any isotropic system greater than 5,000 cP (measured at 0.5 RPM with a Brookfield viscometer, see procedure below) did not mix well on a plant scale with hydrocarbon foaming agents. Even lower viscosity systems, designed to thicken upon addition of hydrocarbon, were not usable in the piston can although they could be dispensed in the bag-in-can dispenser.

Three different methodologies were evaluated for comparative isotropic gel formulations: increasing viscosity with addition of hydrocarbon foaming agent (see e.g. PCT publication no. WO0039273), decreasing or leaving the viscosity unchanged with hydrocarbon foaming agent addition (see e.g. PCT publication no. WO9703646) and the addition of low amounts of soap (see e.g. comparative examples). All were seen to fail in the piston can surprisingly, the inventive lamellar shear-thinning fluid in combination with the same hydrocarbon foaming agent blends in the piston system dispensed well.

Ability to mix with the hydrocarbons and either build or maintain a sufficient viscosity, however, is not sufficient to predict successful dispensing from the piston can. A surprising result of our work is that we found that no matter how viscous the isotropic systems+hydrocarbons were, they failed in the piston can. The specific shear thinning nature of the lamellar systems was found to be important to their success in the piston can.

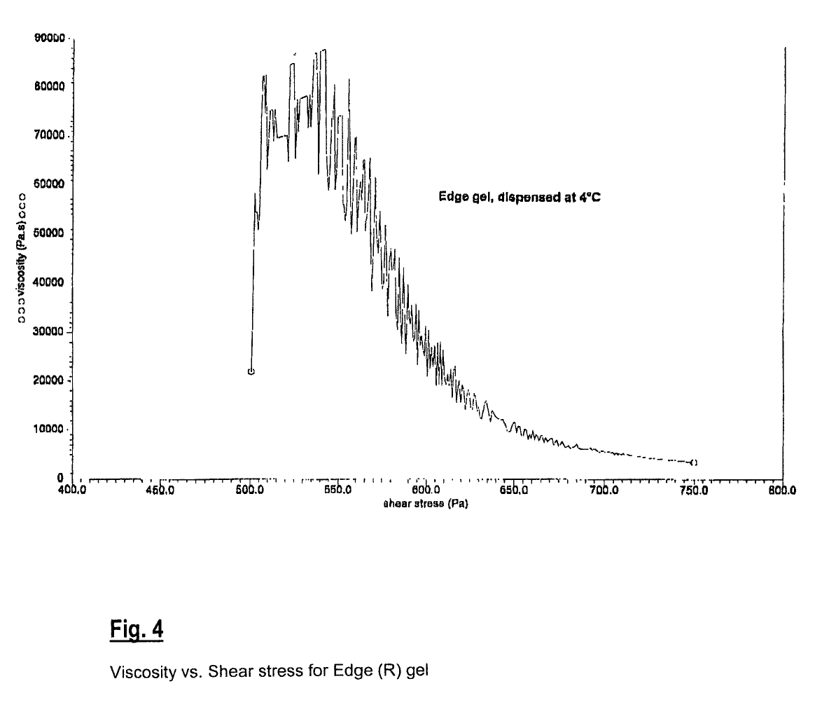

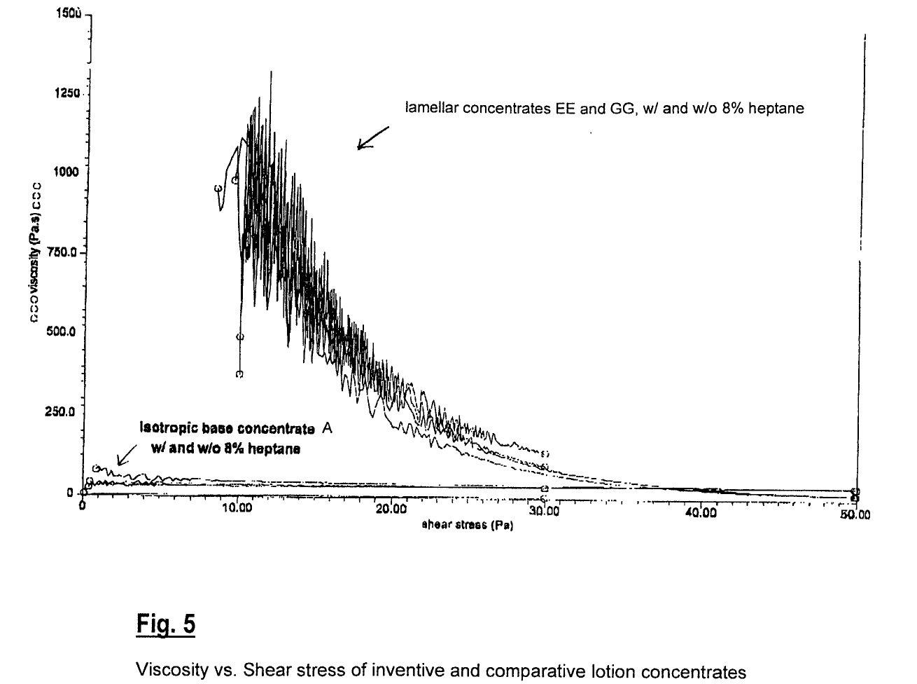

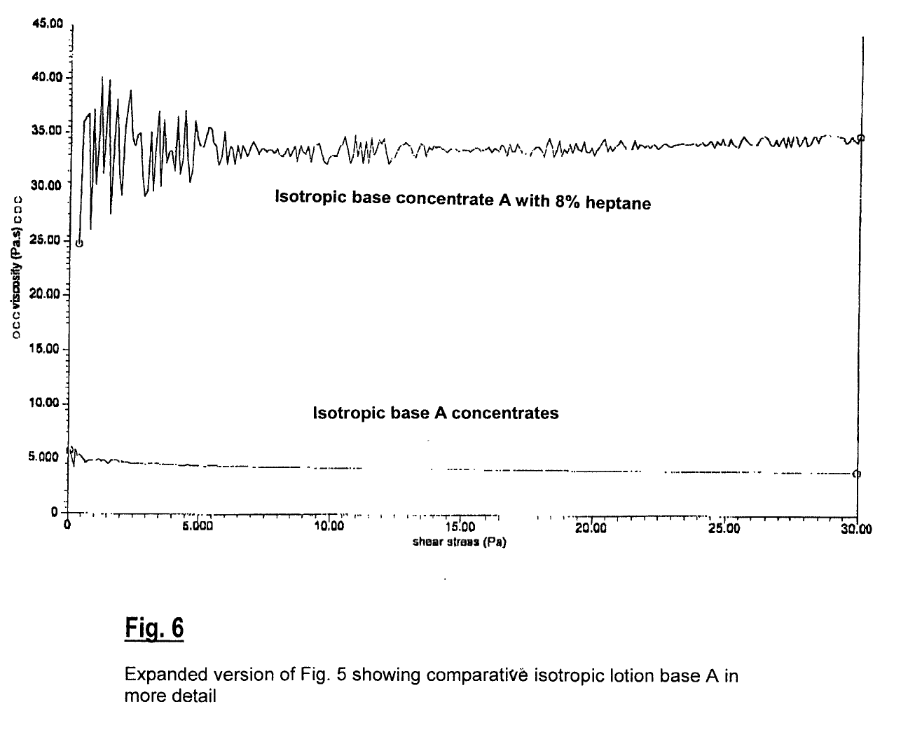

The inventive lamellar systems were found not to separate on mixing with hydrocarbons, nor did they appreciably change viscosity immediately on mixing. To better characterize the lamellar systems, their rheological behavior was evaluated as described below in a controlled stress ramp (see FIGS. 4-6).

A representative selection of isotropic and lamellar samples had the following shear thinning index values (which is defined below):

TABLE 6

| Formula | STI Value |
|---|---|
| A | 0.06 |
| AA | .67 |
| CC | .89 |
| EE | 1.07 |
| LL | 0.68 |

### EXAMPLE 4

### Foam Density

The foam density of the inventive post-foaming lamellar compositions EE and GG was compared to comparative post-foaming isotropic composition A and Bath and Body Works Foam Burst Moisturizing Body Wash in Lavender Flowers and the following mousses: Time Out Mango Ambrosia Whipped Shower®Mousse (Sears), Victoria's Secret Garden Whipped Body Wash® in Whispering Mist®, John Frieda® sheer blonde—blonde ambition® hair mousse; using the method described below.

The density of the inventive lamellar post-foamers within the first 30 seconds of dispensing was found to be between 0.3-0.9 g/ml. After 5 minutes it ranged between 0.2-0.50 g/ml. We compared this to the comparative isotropic post-foaming body wash in the examples and measured the foam density to be as low as 0.1 g/mL immediately on dispensing. It should be noted that the post-foamers all continued to evolve appreciably during the first 5 minutes after dispensing; the mousses either barely evolved, or evolved to a much lower extent, after the 5 minute period.

TABLE 8

| Sample | within 30 sec | after 5 min |
|---|---|---|
| Lamellar EE | 0.72 | 0.46 |
| Lamellar GG | 0.92 | 0.43 |
| Isotropic A | 0.08 | 0.03 |
| Bath and Body FoamBurst ® | 0.53 | 0.27 |
| Time Out Shower ® Mousse | 0.04 | 0.03 |
| Victoria's Secret Garden ® | 0.07 | 0.05 |
| John Frieda ® Hair Mousse | 0.03 | 0.02 |

### EXAMPLE 5

### Oil Holding Capacity

The oil holding capacity of isotropic systems versus lamellar systems were compared using the test method described below.

Three different surfactant bases (all clear, nonlamellar) were used: Suave® unperfumed base, Caress® Wild Blossom body wash, and Softsoap® hydrating body wash with moisture beads (commercially obtained). For each kind of base, 7 samples of 200 g base were added into 600 mL beakers. To the various beakers, aliquots of 1,2,4,6,8,10 wt % sunflower oil were added. A propeller was placed into each beaker and each was mixed at 150 rpm for 5-10 minutes (until appeared uniformly opaque). These were placed into airtight plastic containers for observation at room temperature.

Results:

Complete separation was observed in all Suave ® samples except 1% after 1 week (clear on the bottom with a white creamy layer on top). The 1% sample had a very thin (˜1 mm) white creamy layer on top and was less creamy (but not quite clear) on the bottom. After 2 weeks complete separation was observed. Complete separation was observed in all Caress® samples after 3 days.

After 2 weeks, swirls of translucent gel within the opaque gel were observed in all the Softsoap® samples. This is indicative of the beginning stages of separation.

### EXAMPLE 6

### Foam Stability

The stability of comparative post-foaming isotropic body wash A packaged in a bag-in-can was compared to an inventive post-foaming lamellar composition GG. In this case the comparative sample contained no oil emollients but contained fatty acids which are added as lubricants.

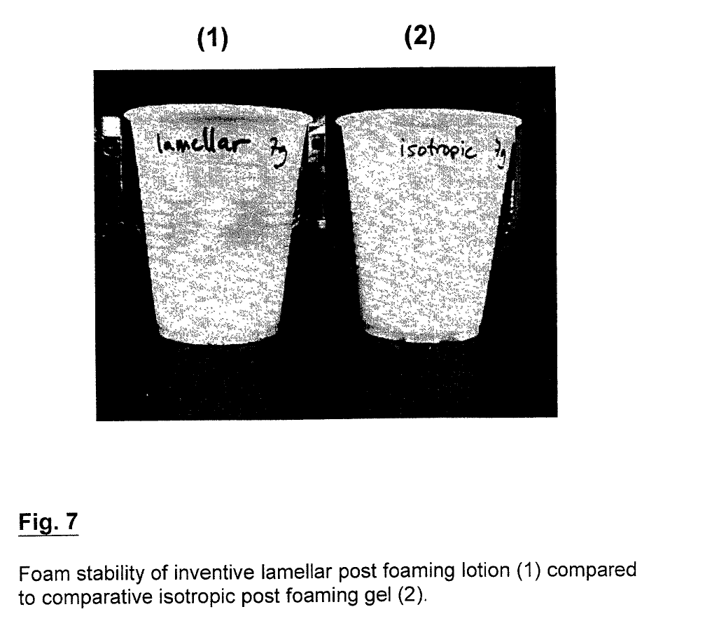

Upon comparing the foam produced (see method below) the inventive lamellar height was 1.5 inches and the isotropic height was 3.5 inches showing decreased and more controlled foaming for the inventive composition (See FIG. 7). Both products contained 8% of a 75/25 isopentane/isobutane blend as the foaming agent.

Once dispensed from bag-in-can type dispensers, it was also observed that in the comparative isotropic systems (such as A), there is “skunktailing”. where the edges of the dispensed stream foam more readily than the center of the stream. This is an indication of improperly dispersed hydrocarbon propellant, and is less observed in the inventive lamellar systems. Foaming of the inventive composition is also delayed compared to comparative post-foamers but can be accelerated with shearing such as the action of a person lathering it on their body.

### Description of Test Methods

1) Skin Moisturization Clinical Test Methodology

The objective of this study was to explore the conditions under which positive moisturization occurs and to compare various inventive and comparative formulations under those conditions. The effects of washing with the various skin cleansing formulations on the dry skin of the lower legs was measured visually and with non-invasive instrumental assessments as described below.

Experimental Method

A randomized, double blind study was used. The study involved a two-day conditioning phase during which the subjects used a cleanser. The test phase of the study consisted of one or two applications of a test material on one or all of the sites with post-treatment visual and instrumental evaluations at 1, 2, 4, 6, 8 and 24 hours after the final application. In this procedure, each outer, lower leg was divided into three sites (upper, middle and lower) and the sites were washed with a designated cleanser. One or two of the six sites remained untreated controls that were included in the randomization scheme as a treatment. The observed effects indicated the point at which skin barrier restoration and moisturization has been achieved.

Twenty (20) subjects between 18-65 years of age in good health were selected who were prone to developing dry skin on their legs in the absence of using a moisturizer.

Subjects refrained from the following: 1) using creams, lotions, moisturizers, bath oils, additives, or any other skin products, other than those provided, on their lower legs for the conditioning phase and during the study; 2) consuming hot or caffeine containing beverages or smoking for one hour prior to instrumental measurements of their skin. 3) using any appliances, (wash cloth, sponge, etc.) on the test sites for duration of the study, and refrained from shaving their legs 30 hours before a scheduled appointment throughout the study, 4) wetting the test sites within 3 hours of evaluation; 5) excessive sun exposure (resulting in sunburn) on the lower legs during the conditioning and treatment phases of the study.

All subjects have dryness scores of <3.0 and erythema scores of <1.0 on the test sites, and be free of cuts or abrasions on the outer, lower legs to be included into the conditioning phase of the study; and, have dryness scores of at least 1.0 but no greater than 2.5 with a maximum of 1.0 difference among all sites, and erythema scores of <1.0 following the conditioning phase to be included in the test phase of the study.

For each subject, each outer, lower leg was divided into three sites, 2.5 by 2.5 inch squares (upper, middle and lower) for a total of 6 test sites per subject. One or two of the sites was left untreated and included in the randomization of products. For studies utilizing instruments, baseline instrumental measurements were taken on each test site. The baseline measurements for the sites must be within 100 units of each other for Skicon readings, within 15 units of each other for the Corneometer and Evaporimeter values to be included in the study.

The sites were treated once or twice with the designated amount of test material for 10 seconds. Cleansing products remained on the test sites for a maximum of 90 seconds. Sites were rinsed for 30 seconds each, ensuring the test material from one site did not contaminate another site. After rinsing, the test sites were gently dried with a paper towel. The application consists of dosing with up to 5 different test materials on the designated sites, one material per test site, and one or two untreated sites. The following wash procedure was performed:

1. The test site is wet on the outer, lower leg with warm water (92°±4° F.).

2. Fingers are wetted and the test material is dispensed to the test site.

3. Application is made by gently gliding fingers from side to side over the designated test site for the designated period of time at a rate of approximately one stroke/second (a stroke is from front to back of leg and then again to the front) ensuring not to go outside of the designated test site. Equal pressure is applied to all test sites. The sites may be washed simultaneously.

4. The test site is rinsed with warm water, patted dry with a soft disposable towel and the procedure is repeated for the other test sites. When benefit agents or moisturizers are being tested, they will not be rinsed off the site or dried with a towel but will be allowed to air-dry for a minimum of two minutes.

Evaluation Methods

Visual Assessment

The scale as shown in Table I was used to assess the test sites for dryness, and is a 0 to 4 scale with half-point increments. Initial visual assessments were made prior to the conditioning phase to ensure that none of the subjects exceed the maximum dryness and erythema scores set for inclusion.

TABLE 1

| Grade | Dryness Scale | Erythema Scale |
|---|---|---|
| 0.0 | No dryness | No erythema |
| 0.5 | Perceptible dryness, fine white lines | |
| 1.0 | Fine dry lines, white powdery look and/or | Mild erythema |
| | some uplifting flakes, on less than 30% of the | |
| | test site | |
| 1.5 | More uniform flaking, covering 30-50% of | |
| | the test site | |
| 2.0 | Uniform, marked flaking covering more | Moderate |
| | than 50% of the test site area and/or isolated | confluent |
| | scaling | Erythema |
| 2.5 | Slight to moderate scaling | |
| 3.0 | Moderate to severe scaling with some | Marked |
| | uplifting of the scales | erythema |
| 3.5 | Severe scaling and/or slight fissuring | |
| 4.0 | Severe scaling and severe fissuring | Deep erythema |

Baseline visual assessments were made prior to the start of the product application phase and thereafter, immediately before each of the instrumental assessments, to evaluate skin dryness and erythema. One trained evaluator conducted all visual evaluations during the product application phase. The evaluator examined both lower legs with the aid of an illuminated magnifying lamp with a 3 diopter lens and a shadow-free circular cool white fluorescent light source. Half-point increments for erythema were used for responses not warranting a full point increase. To maintain the evaluator's blindness to product assignment, the visual assessments were conducted in a separate area away from the product application area.

An endpoint score was reached when a subject develops an erythema or dryness grade of 3.0 or higher on any test site. Any condition more than a 4.0 was assigned a 4.0.

Instrumental Assessment

All Instrumental evaluations were taken following a 30-minute acclimation period. Indoor humidity and temperate was recorded. Instrumental measurements were taken at some or all of the following time points: 0, 1, 2, 4, 6, 8 and 24 hours after product application. Instruments used for this protocol include: ServoMed Evaporimeter with EP1 or EP2 probe, Corneometer

CM820 and the Skicon Skin Hygrometer with the MT-8C probe. The room temperature was maintained at 68° to 77° F. and 30% to 40% Relative Humidity.

Within Test Product Effects

The effects of each test product and the untreated site were examined by comparing the clinical grade at each time point versus the baseline clinical grade using the Wilcoxon Signed-Rank test, Pratt-Lehmann Version, as documented in Lehmann, E. L., Nonparametrics Statistical Methods Based on Ranks, pg. 123, Holden-Day, Inc., and McGraw-Hill Book Co., 1975. Statistical significance was determined at the 90% confidence level (p 0. 10).

Between Test Product Effects

At each evaluation point, the Pratt/Lehmann Version of the Wilcoxon Signed-Rank test was conducted on the difference in clinical grades (evaluation—baseline.) for each pair of treatments using the subject as a block. The untreated site was considered as a treatment in this analysis, indicating comparisons of the treatments within a group.

For the instrumental analysis data, the same comparisons were made using parametric statistical methods. The TEWL, conductance and capacitance measurements were averaged separately for each subject, site and session. For all treatments, treatment differences were statistically compared using an analysis of variance with the subject as a block. If overall statistical significant differences were detected (p<0.10), pairwise treatment comparisons were implemented by comparing the least square means using Fisher's Least Significant Difference.

The following references are herein included in their entirety: 1) K. D. Ertel, G. H. Keswick, and P. B. Bryant: “Forearm Controlled Application Technique for Estimating the Relative Mildness of Personal Cleansing Products”, J. Soc. Cosmet Chem., 46, 67-76, 1995. 2) M. F Lukacovic, F. E. Dunlap, S. E. Michaels, M. O. Visscher, and D. D. Watson: “Forearm Wash Test to Evaluate the Mildness of Cleansing Products”, J.Soc. Cosmet. Chem., 39, 355-366, 1988. 3) P. T. Sharko, R. I. Murahata, J. J. Leyden, and G. I. Grove: “Ann Wash with Instrumental Evaluation—A Sensitive Technique for Differentiating the Initiation Potential of Personal Washing Products”, J. Dermalclinical Eval. Soc., 2, 19-27, 1991. 4) G. W. Snedecor and W. G. Cochran: Statistical Methods. Ames, Iowa: The Iowa State University Press, pp. 84-86, 1980.

2) Aerosol Can Filling Procedure:

The neat cleansing lotion is prepared for filling into aerosol piston cans. Manual filling consists of chilling the product and the volatile foaming agent(s) to 0° C., mixing the two in a beaker (e.g. 8% pure isopentane or mixtures of isopentane and isobutane or any suitable foaming agent) then adding to the can, and crimping the valve on the can. Propellant is gassed through the bottom with commercially available propellants such as A31 or A46. The numerical designation reflects the approximate vapor pressure in psig. Such vapor pressures can be achieved for example using the following in various combinations: isobutane, n-butane, and propane, and the like. Production filling may utilize in line blending technology of the two components before they reach the filling machine.

For filling into bag-in-can, the cans are first pressurized (using for example an “Undercup” Crimper P 2002-500 available from Pamasol, Switzerland) with compressed air at 2.5 bar, or a liquifiable volatile hydrocarbon or other suitable propellant material and sealed by insertion of the valve/diptube/laminated bag assembly before adding the lotion base-foaming agent mixture. Base product and foaming agent are filled into the bag through the valve in a single operation by use of a machine designed for such purpose, such as the KP Aerofill System from Kartridge Pak.

3) Rheological Test Methods:

A. Controlled Shear Stress

Viscosity data for a stress sweep was collected using a Carri-Med CSL-100 rheometer. The experiments were run in controlled temperature, shear stress ramp between two inputted shear stress values. A cone-in-plate geometry was used with a stainless steel 4 cm 2 degree cone.

Procedure: Power on the rheometer. Verify that the “house” air via an air filter is supplying the air bearing. Remove protective cover over the air bearing. With the set screw, attach the stainless 4 cm 2 degree cone to finger tight. Set the measurement temperature to 25C and autozero the gap, after which the gap will be set to the geometry gap of 48 micrometers.

Measurements were performed at 4° C. in order to characterize samples that have already been mixed with hydrocarbons that are volatile at room temperature. To accomplish this, cans of the products of interest are placed in 4° C. refrigerators overnight. The measurement temperature is inputted into the rheometer, the cone is refrigerated until immediately before using, and autozeroing of the gap with the cone and plate is done at 4° C.

Lower the plate and place about 1 cc of sample on the plate using a Teflon spatula. Raise the plate to meet the cone such that there will be minimal overflow of sample around the outside of the cone. Place a cover around the sample measurement area to prevent excessive moisture loss.

Set the experimental conditions (e.g. from 0-30 Pa to be sampled in 5 minutes) and begin the experiment. Note that for samples with high yield tress (which is usually the case with the inventive lamellar samples) data may not be able to be collected at the lower shear stress values. The Rheometer will automatically begin collecting data at the lowest shear stress at which it can achieve a certain minimum angular velocity.

Viscosities at 10 and 30 Pa are used to calculate the Shear Thinning Index, or STI:

STI=log [viscosity at 10 Pa/viscosity at 30 Pa].

B. Controlled Shear Rate

As a routine laboratory benchmark, viscosities are measured at 0.5 RPM using T-bar spindle A. Apparatus: Brookfield RVT viscometer with Helipath Accessory; chuck, weight and closer assembly for T-bar attachment; T-bar spindle A; plastic cups diameter greater than 6.35 cm (2.5 inches).

Procedure: Verify that the viscometer and the helipath stand are level by referring to the bubble levels on the back of the instrument. Connect the chuck/closer/weight assembly to the Viscometer (note the left-hand coupling threads). Clean Spindle A with deionized water and pat dry with a Kimwipe sheet. Slide the spindle in the closer and tighten. Set the rotational speed at 0.5 RPM. In case of a digital viscometer (DV), select the % mode and press autozero with the motor switch on. Place the product in a plastic cup with inner diameter of greater than 6.35 cm (2.5 inches). The height of the product in the cup should be at least 7.6 cm (3 inches). The temperature of the product should be 25° C. Lower the spindle into the product (˜6.4 mm or 4 inches). Set the adjustable stops of the helipath stand so that the spindle does not touch the bottom of the plastic cup or come out of the sample. Start the viscometer and allow the dial to make one or two revolutions before turning on the helipath stand. Note the dial reading as the helipath stand passes the middle of its downward traverse. Multiply the dial reading by a factor of 4,000 and report the viscosity reading in cps.

4) Foam Density Determination Method

Foam density was measured using a stainless steel pycnometer. First, the pycnometer was rinsed with tap water followed by distilled water. It was rinsed with acetone, dried, and allowed to come to room temperature. The empty pycnometer was tared. To calculate the volume of the pycnometer, the body of the pycnometer was carefully filled with water, avoiding air bubbles. The cover was placed on and collar screwed into place. Excess water was wiped away and the filled pycnometer was weighed.

After cleaning and drying, the product was added to the pycnometer and the cover was carefully pushed down until seated. Excess sample expelled through the center was wiped away and the collar was screwed on. Excess samples was cleaned from the outside of the pycnometer and the filled pycnometer was weighed. Density is calculated using the following equation:

ρ=g sample/g H₂O.

5) Foam Comparison Procedure:

Sample one lamellar and one isotropic post-foaming composition each with 8% foaming agent (75/25 blend of isopentane and isobutane). Dispensed 7 g of each product into separate 7 oz cups simultaneously. After 5 minutes, the height of the foam was measured. A visual evaluation for foam quality was also done.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

TABLE 4
Lamellar Formulations

| | wt (%) | | | | | | | | | | | |
|---|---|---|---|---|---|---|---|---|---|---|---|---|
| INCI name | AA | BB | CC | DD | EE | FF | GG | HH | II | JJ | KK | LL |
| alkyl polyglucoside | 10.0 | 0.0 | 0.0 | 0.0 | 0.0 | 0.0 | 0.0 | 0.0 | 0.0 | 0.0 | 0.0 | 0.0 |
| sodium | 0.0 | 0.0 | 0.0 | 0.0 | 0.0 | 5.7 | 0.0 | 0.0 | 0.0 | 0.0 | 0.0 | 0.0 |
| lauroamphoacetate | | | | | | | | | | | | |
| sodium laureth sulfate | 5.0 | 12.3 | 12.3 | 12.3 | 12.3 | 12.3 | 12.3 | 12.3 | 12.3 | 11.0 | 13.0 | 12.4 |
| Cocoamidopropyl | 5.0 | 5.7 | 5.7 | 5.7 | 5.7 | 0.0 | 5.7 | 5.7 | 5.7 | 6.0 | 5.0 | 5.7 |
| betaine | | | | | | | | | | | | |
| Cocamide MEA | 1.9 | 1.9 | 1.9 | 1.9 | 1.9 | 1.9 | 1.9 | 1.9 | 1.9 | 1.5 | 2.0 | 2.3 |
| citric acid | 0.0 | 0.0 | 0.2 | 0.2 | 0.0 | 0.6 | 0.0 | 0.2 | 0.0 | 0.0 | 0.0 | 0.0 |
| lauric acid | 2.7 0.0 | 2.3 | 0.0 | 3.8 | 2.7 | 2.3 | 2.3 | 2.3 | 3.3 | 3.8 | 3.7 | |
| isostearic acid | 0.0 | 5.0 | 0.0 | 5.0 | 0.0 | 0.0 | 0.0 | 0.0 | 0.0 | 0.0 | 0.0 | 0.0 |
| oleic acid | 0.0 | 0.0 | 0.0 | 5.0 | 0.0 | 0.0 | 0.0 | 0.0 | 0.0 | 0.0 | 0.0 | 0.0 |
| PEG-30 | 0.0 | 0.0 | 0.0 | 0.0 | 0.3 | 0.0 | 0.0 | 0.0 | 0.0 | 0.3 | 0.3 | 0.0 |
| dipolyhydroxystearate | | | | | | | | | | | | |
| Guar hydroxypropyl | 0.5 | 0.6 | 0.6 | 0.6 | 0.2 | 0.7 | 0.7 | 0.2 | 0.7 | 0.0 | 0.0 | 0.5 |
| trimonium chloride | | | | | | | | | | | | |
| polyquaternium-10 | 0.0 | 0.0 | 0.0 | 0.0 | 0.0 | 0.0 | 0.0 | 0.0 | 0.0 | 0.6 | 0.0 | 0.0 |
| polyquaternium-7 | 0.0 | 0.0 | 0.0 | 0.0 | 0.0 | 0.0 | 0.0 | 0.0 | 0.0 | 0.0 | 0.3 | 0.0 |
| isopropyl palmitate | 1.5 | 1.5 | 1.5 | 1.5 | 1.5 | 1.5 | 1.5 | 1.5 | 0.0 | 1.5 | 1.5 | 2.0 |
| sunflower seed oil | 16.0 | 16.0 | 0.0 | 7.0 | 16.0 | 21.3 | 21.3 | 16.0 | 21.3 | 16.0 | 17.0 | 14.0 |
| petrolatum | 5.0 | 3.7 | 5.0 | 0.0 | 3.7 | 3.7 | 3.7 | 3.7 | 3.7 | 5.0 | 4.0 | 3.3 |
| lanolin alcohol | 0.5 | 0.5 | 0.5 | 0.0 | 0.5 | 0.5 | 0.5 | 0.5 | 0.5 | 0.8 | 0.0 | 0.0 |
| dimethicone | 0.0 | 0.0 | 16.0 | 0.0 | 0.0 | 0.0 | 0.0 | 0.0 | 0.0 | 0.0 | 0.0 | 0.0 |
| castor oil | 0.0 | 0.0 | 0.0 | 9.0 | 9.0 | 0.0 | 0.0 | 0.0 | 0.0 | 0.0 | 0.0 | 0.0 |
| glycerin | 1.0 | 5.7 | 1.0 | 1.0 | 5.7 | 5.7 | 5.7 | 5.7 | 5.7 | 2.0 | 6.0 | 1.0 |
| fragrance | 1.0 | 1.0 | 1.0 | 1.0 | 1.0 | 1.0 | 1.0 | 1.0 | 1.0 | 1.0 | 1.0 | 1.3 |
| dyes | 0.0 | 0.0 | 0.0 | 0.0 | 0.0 | 0.0 | 0.0 | 0.0 | 0.0 | 0.0 | 0.0 | 0.1 |
| preservative | 0.1 | 0.1 | 0.1 | 0.1 | 0.1 | 0.1 | 0.1 | 0.1 | 0.1 | 0.1 | 0.1 | 0.1 |
| DI water | to 100 | to 100 | to 100 | to 100 | to 100 | to 100 | to 100 | to 100 | to 100 | to 100 | to 100 | to 100 |
| All were prepared as follows: | | | | | | | | | | | | |
| All lipophilic ingredients, lamellar structurants, cocamide MEA, and lamellar stabilizer (if using) were mixed at 150-180° F., setting aside 5-7% of the triglyceride oil or mineral oil. At this point the heating is turned off. This was followed by the addition of the glycerin, then about 1/3 of the remaining water, then the anionic surfactants, the amphoteric/zwitterionics, then the rest of the water. Isopropyl palmitate is added next, followed by the addition of a slurry made of the cat- | | | | | | | | | | | | |
| #ionic polymer with the reserved oil. Once the mixture reaches 120° F., the preservatives are added, and once it cools to 100° F., the dyes (if using) and fragrance are added. | | | | | | | | | | | | |

TABLE 5
Isotropic Formulations

| | wt % | | | | | | | | | | | | | | | | | |
|---|---|---|---|---|---|---|---|---|---|---|---|---|---|---|---|---|---|---|
| INCI name | A | B | C | D | E | F | G | H | I | J | K | L | M | N | P | Q | R | S |
| Sodium Laureth Sulfate | 12.5 | 9.3 | 13.7 | 12.5 | 11.07 | 12.5 | 13.7 | 9.3 | 12.5 | 9.3 | 12.5 | 12.5 | 13.7 | 12.5 | 10 | 9.3 | 12 | 9.3 |
| Cocamidopropyl betaine | 5 | 2.5 | 0.8 | 3 | 4.43 | 3 | 3.3 | | 3 | 2.5 | 3 | 5 | 3.3 | 3 | | | 4.5 | |
| Sodium Cocoyl Isothionate | | 5 | | | | | | 5 | | 5 | | | | | 5 | 5 | | 5 |
| Sodium Lauryl Sulfate | | | | | | | | | | | | | | | | | 1 | |
| Cocamide-MEA | | 1.2 | 0.5 | 1 | 1 | 1 | 0.5 | | 1 | 1.2 | 0.5 | | 0.5 | 0.5 | | | 1 | 1.2 |
| Cocamide DEA | | | | | | | | | | | | | | | | 1.2 | | |
| Potassium Cocoate | | | | | | | | 2.5 | | | | | | | 4 | | | |
| Hydroxypropyltrimonium | 0.15 | | 0.2 | 0.25 | 0.25 | 0.25 | 0.25 | | 0.25 | | 0.2 | | 0.25 | 0.2 | | | | |
| chloride | | | | | | | | | | | | | | | | | | |
| Hydroxypropyl guar | | 0.4 | | | | | | 0.4 | | 0.4 | | | | | 0.4 | 0.4 | 0.2 | 0.4 |
| Hydroxypropyltrimonium | | | | | | | | | | | | | | | | | | |
| chloride | | | | | | | | | | | | | | | | | | |
| Cocamidopropyl betaine | | | 6.6 | | | | | | | | | | | | | 6.6 | | 6.6 |
| and Glyceryl Monolaurate | | | | | | | | | | | | | | | | | | |
| Glyceryl Monolaurate | | 1.5 | | | | | | 1.65 | | 1.5 | | | | | 1.65 | | 1 | |
| PEG-120 Methyl Glucoate | 1.32 | | 1 | | 1.32 | 1 | 1 | | | | | 1.32 | | | | | | |
| Dioleate | | | | | | | | | | | | | | | | | | |
| PEG-120 Methyl Glucose | | | | 0.75 | | | | | | | | | | | | | | |
| Trioleate | | | | | | | | | | | | | | | | | | |
| PEG-150 Pentaerylthrityl | | | | | | | | | | | 0.5 | | | | | | | |
| tetrastearate | | | | | | | | | | | | | | | | | | |
| Glyceryl Palmate + PEG-7 | | | | | | | | | | | | | | 2 | | | | |
| Glyceryl cocate | | | | | | | | | | | | | | | | | | |
| Acrylates Copolymer | | | | | | 0.5 | | | | | | | | | | | | |
| Sodium Cocoyl Glutamate | | 0.8 | | | | | | 0.8 | | 0.8 | | | 1 | | 2 | 0.8 | | 0.8 |
| Stearic Acid | | | | | | | | | | | | | | | | | | |
| Hydrolyzed Wheat Protein | | | 0.23 | 0.23 | | | | 0.23 | 0.23 | | | 0.23 | | | | | | |
| Isopropyl Palmitate | 1.5 | | 1.5 | 1.5 | 1.5 | 1.5 | 1.5 | | 1.5 | | 1.5 | 1.5 | 1.5 | 1.5 | | | | |
| PEG-40 Hydrogenated | 0.5 | | 0.5 | 0.5 | 0.5 | 0.5 | 0.5 | | 0.5 | | 0.5 | 0.5 | 0.5 | 0.5 | | | | |
| Castor Oil | | | | | | | | | | | | | | | | | | |
| Propylene Glycol | | | 0.5 | | | | | 0.5 | | | 0.5 | | | 0.5 | 0.5 | 0.5 | 0.5 | 0.5 |
| Glycerin | | | | 0.5 | 0.5 | 0.5 | | | 0.5 | | 0.5 | | | 0.5 | | | | |
| Fragrance | 0.9 | 0.9 | 0.9 | 0.9 | 0.9 | 0.9 | 0.9 | 0.9 | 0.9 | 0.9 | 0.9 | 0.9 | 0.9 | 0.9 | 0.9 | 0.9 | 0.9 | 0.9 |
| Color | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 | 0.0006 |
| Preservative | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 | 0.0003 |
| Citric Acid | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| Water | to 100 | to 100 | to 100 | to 100 | to 100 | to 100 | to 100 | to 100 | to 100 | to 100 | to 100 | to 100 | to 100 | to 100 | to 100 | to 100 | to 100 | to 100 |
| Viscosity | 688 | 9038 | 25000+ | 835 | 63400 | 2677 | 9840 | 393 | 344 | 24460 | 221 | 197 | 0 | 442 | 197 | 47800 | 77020 | 25000+ |
| Mix with Heptane? | YES | YES | YES | YES | NO | YES | YES | YES | YES | YES | YES | YES | YES | YES | YES | YES | NO | YES |
| Viscosity after 6% Heptane | 7785 | 123 | 5108 | 5157 | | 25000+ | 25000+ | 0 | 147 | 835 | 2357 | 8854 | 196 | 418 | 0 | 295 | | 712 |
| addition | | | | | | | | | | | | | | | | | | |
| Process | 6 | 8 | 9 | 6 | 3 | 7 | 2 | 12 | 1 | 8 | 10 | 4 | 2 | 5 | 12 | 8 | 11 | 8 |

TABLE 7
Processes used to formulate isotropic samples in table 5

| Process 1 | Process 2 | Process 3 |
|---|---|---|
| Add initial water charge and | Add initial water charge and | Add initial water charge and |
| heat to 150 F.-160 F. | heat to 150 F.-160 F. | heat to 150 F.-160 F. |
| Premix cationic polymer with | Add anionic surfactant | Premix cationic polymer with |
| glycerin and add to main | | glycerin and add to main |
| batch | | batch |
| Mix 15 minutes | Add amphoteric surfactant | Mix 15 minutes |
| Add anionic surfactant | Premix cationic polymer with | Add anionic surfactant |
| | Isopropyl Palmitate and add | |
| | to main batch | |
| Add amphoteric surfactant | Mix 15 minutes | Add amphoteric surfactant |
| Add other ingredients such | Add other ingredients such | Add other ingredients such |
| as PEG-150 Distearate, | as PEG-150 Distearate, | as PEG-150 Distearate, |
| Cocamide-MEA, Hydrolyzed | Cocamide-MEA, Hydrolyzed | Cocamide-MEA, Hydrolyzed |
| Wheat Protein, Glycerin, or | Wheat Protein, Glycerin, or | Wheat Protein, Glycerin, or |
| Isopropyl Palmitate | Isopropyl Palmitate | Isopropyl Palmitate |
| Maintain heat and mix for 30 | Add PEG-120 Methyl | Add PEG-120 Methyl |
| minutes | Glucose Dioleate | Glucose Dioleate |
| Begin to cool to 95F | Maintain heat and mix for 30 | Maintain heat and mix for 30 |
| | minutes | minutes |
| Premix Fragrance, PEG-40 | Begin to cool to 95 F. | Begin to cool to 95 F. |
| Hydrogenated Castor Oil, | | |
| and Isopropyl Palmitate and | | |
| add to main batch at 110 F. | | |
| Mix for 15 minutes | Premix Fragrance and PEG- | Premix Fragrance and PEG- |
| | 40 Hydrogenated Castor Oil | 40 Hydrogenated Castor Oil |
| | and add to main batch at | and add to main batch at |
| | 110 F. | 110 F. |
| Add preservatives, color, and | Add preservatives, color, | Add preservatives, color, and |
| promotionals | fragrance and promotionals | promotionals below 110 F. |
| | below 110 F. | |
| Add citric acid to adjust pH to | Add citric acid to adjust pH | Add citric acid to adjust pH to |
| a target range of 5.5-6.0 | to a target range of 5.5-6.0 | a target range of 5.5-6.0 |
| Mix for 15 minutes and | Mix for 15 minutes and | Mix for 15 minutes and |
| measure final viscosity | measure final viscosity | measure final viscosity |
| Process 4 | Process 5 | Process 6 |
| Add initial water charge | Add initial water charge and | Add initial water charge and |
| and heat to 150 F.-160 F. | heat to 150 F.-160 F. | heat to 150 F.-160 F. |
| Add anionic surfactant | Premix cationic polymer with | Premix cationic polymer with |
| Add amphoteric surfactant | Propylene glycol and add to | Isopropyl Palmitate and add |
| | main batch | to main batch |
| | Mix 15 minutes | Mix 15 minutes |
| Add Isopropyl Palmitate | Add amphoteric surfactant | Add anionic surfactant |
| Mix 15 minutes | Add anionic surfactant | Add amphoteric surfactant |
| Add PEG-120 Methyl | Add other ingredients such | |
| Glucose Dioleate | as PEG-150 Distearate, | |
| | Cocamide-MEA, Hydrolyzed | |
| | Wheat Protein, Glycerin, or | Add PEG-120 Methyl |
| | Isopropyl Palmitate | Glucose Dioleate |
| Maintain heat and mix for | Add Glyceryl Laurate or | |
| 30 minutes | other low molecular weight | Maintain heat and mix for 30 |
| | polymer | minutes |
| Begin to cool to 95 F. | Maintain heat and mix for 30 | Begin to cool to 95 F. |
| | minutes | |
| Premix Fragrance and | Begin to cool to 95 F. | Premix Fragrance and PEG |
| PEG-40 Hydrogenated | | 40 Hydrogenated Castor Oil |
| Castor Oil and add to main | | and add to main batch at |
| batch at 110 F. | | 110 F. |
| Add preservatives, color, | Premix Fragrance and PEG- | Add preservatives, color, and |
| and promotionals below | 40 Hydrogenated Castor Oil | promotionals below 110 F. |
| 110 F. | and add to main batch at | |
| | 110 F. | |
| Add citric acid to adjust pH | Add preservatives, color, | Add citric acid to adjust pH to |
| to a target range of 5.5-6.0 | and promotionals below | a target range of 5.5-6.0 |
| | 110 F. | |
| Mix for 15 minutes and | Add citric acid to adjust pH | Mix for 15 minutes and |
| measure final viscosity | to a target range of 6.0-6.5 | measure final viscosity |
| | Mix for 15 minutes and | |
| | measure final viscosity | |
| Process 7 | Process 8 | Process 9 |
| Add initial water charge and | Add initial water charge and | Add initial water charge and |
| heat to 150 F.-160 F. | heat to 150 F.-160 F. | heat to 150 F.-160 F. |
| Add Acrylates Copolymer | Add first anionic surfactant | Premix cationic polymer with |
| | | Propylene glycol and add to |
| | | main batch |
| Add anionic surfactant | Add second anionic | Mix 15 minutes |
| | surfactant | |
| Add amphoteric surfactant | Add amphoteric surfactant | Add amphoteric surfactant |
| Premix cationic polymer with | Add cationic polymer | Add low molecular weight |
| Isopropyl Palmitate and add | | polymer |
| to main batch | | |
| Mix 15 minutes | Add Glyceryl Laurate or | Add anionic surfactant |
| | other low molecular weight | |
| | polymer | |
| Add other ingredients such | Add Sodium Cocoyl | Add other ingredients such as |
| as PEG-150 Distearate, | Glutamate | PEG-150 Distearate, |
| Glycerin, and Cocamide- | | Cocamide-MEA, Hydrolyzed |
| MEA | | Wheat Protein, Glycerin, or |
| | | Isopropyl Palmitate |
| Maintain heat and mix for 30 | Add Cocamide-MEA | Add PEG-120 Methyl |
| minutes | | Glucose Dioleate |
| Begin to cool to 95 F. | Begin to cool to 95 F. | Maintain heat and mix for 30 |
| | | minutes |
| Premix Fragrance and PEG- | Add preservatives, color, | Begin to cool to 95 F. |
| 40 Hydrogenated Castor Oil | fragrance, and promotionals | |
| and add to main batch at | below 110 F. | |
| 110 F. | | |
| Add preservatives, color, and | Add citric acid to adjust pH to | Premix Fragrance and PEG- |
| promotionals below 110 F. | a target of 6.4 | 40 Hydrogenated Castor Oii |
| | | and add to main batch at |
| | | 110 F. |
| Add citric acid to adjust pH to | Mix for 15 minutes and | Add preservatives, color, and |
| a target range of 5.5-6.0 | measure final viscosity | promotionals below 110 F. |
| Mix for 15 minutes and | | Add citric acid to adjust pH to |
| measure final viscosity | | a target range of 5.5-6.0 |
| | | Mix for 15 minutes and |
| | | measure final viscosity |
| Process 10 | Process II | Process 12 |
| Add initial water charge | Add initial water charge and | Add initial water charge and |
| and heat to 150 F.-160 F. | heat to 150 F.-160 F. | heat to 150 F.-160 F. |
| Premix cationic polymer | Premix cationic polymer with | Add first anionic surfactant |
| with Propylene glycol and | Propylene glycol and add to | |
| add to main batch | main batch | |
| Mix 15 minutes | Mix 15 minutes | Add second anionic |
| | | surfactant |
| Add amphoteric surfactant | Add amphoteric surfactant | Add Potassium Cocoate |
| Add anionic surfactant | Add anionic surfactant | Premix cationic polymer with |
| | | Propylene glycol and add to |
| | | main batch |
| Add other ingredients such | Add second anionic | Add low molecular weight |
| as PEG-150 Distearate, | surfactant | polymer |
| Cocamide-MEA, | | |
| Hydrolyzed Wheat Protein, | | |
| Glycerin, Isopropyl | | |
| Palmitate, or low | | |
| molecular weight polymer | | |
| Add long chain PEG | Add other ingredients such | Add Sodium Cocoyl |
| | as PEG-150 Distearate, | Glutamate |
| | Cocamide-MEA, Hydrolyzed | |
| | Wheat Protein, Glycerin, | |
| | Isopropyl Palmitate, or low | |
| | molecular weight polymer | |
| Maintain heat and mix for | Maintain heat and mix for 30 | Begin to cool to 95 F. |
| 30 minutes | minutes | |
| Begin to cool to 95 F. | Begin to cool to 95 F. | Add preservatives, color, |
| | | fragrance, and promotionals |
| | | below 110 F. |
| Premix Fragrance and | Add preservatives, color, | Add citric acid to adjust pH to |
| PEG-40 Hydrogenated | fragrance, and promotionals | a target of 7.3 |
| Castor Oil and add to main | below 110 F. | |
| batch at 110 F. | | |
| Add preservatives, color, | Add citric acid to adjust pH | Mix for 15 minutes and |
| and promotionals below | to a target range of 6.0-6.5 | measure final viscosity |
| 110 F. | | |
| Add citric acid to adjust pH | Mix for 15 minutes and | |
| to a target range of 6.0-6.5 | measure final viscosity | |
| Mix for 15 minutes and | | |
| measure final viscosity | | |

We claim:

1. A liquid cleansing and moisturizing composition comprising:
(a) from about 80 to about 97% by wt. of a neat cleansing lotion having
about 0.5 to about 65% by wt. of the total composition of at least one non-soap anionic or mixture of non-soap anionic surfactants;
about 35 to about 90% by wt. of the total composition of water;
(b) from about 3 to about 20% by wt. of the total composition of at least one volatile foaming agent or mixture thereof; and
wherein the neat cleansing lotion is a lamellar structured shear thinning composition at 25 C.

2. The composition of claim 1 wherein the at least one non-soap anionic or mixture of non-soap anionic surfactants is in the concentration range of about 1 to about 25% by wt. of the total composition.

3. The composition of claim 1 wherein the neat cleansing lotion further comprises about 0.1 to about 25% by wt. of the total composition of a surfactant selected from amphoteric, zwitterionic or mixtures thereof.

4. The composition of claim 1 wherein the neat cleansing lotion further comprises about 0.5 to about 50% by wt. of the total composition of lipophilic emollients, humectants, and mixtures thereof.

5. The composition of claim 4 wherein the neat cleansing lotion comprises about 6 to about 35 by wt. of the total composition of lipophilic emollients, humectants, and mixtures thereof.

6. The composition of claim 4 wherein the neat cleansing lotion contains at least one lipophilic emollient in a concentration greater than about 10%.

7. The composition of claim 6 wherein the neat cleansing lotion contains at least one lipophilic emollient in a concentration greater than about 12%.

8. The composition of claim 6 wherein the at least one lipophilic emollient is a triglyceride oil.

9. The composition of claim 1 wherein the neat cleansing lotion contains
About 0.1% to about 15% by wt. of the total composition of a lamellar phase inducing structurant selected from:
C8 to C24 alkenyl or branched alkyl fatty acid or ester thereof with a melting point below 25C;
C8 to C₂₄ alkenyl or branched alkyl fatty alcohol or ether thereof with melting point below 25C;
C5 to C12 alkyl fatty acids; and
Hydroxystearin.

10. The composition of claim 1 wherein the neat cleansing lotion has a shear thinning index greater than about 0.4.

11. The composition of claim 10 wherein the neat cleansing lotion has a shear thinning index greater than about 0.5.

12. The composition of claim 4 wherein the lipophilic emollient is selected from a triglyceride oil, mineral oil, petrolatum, and a blend thereof; and the humectants are selected from polyhydric alcohols, polyols, and blends thereof.

13. The composition of claim 1 further comprising at least one cationic skin conditioning agent.

14. The liquid cleansing and moisturizing composition of claim 13 wherein the cationic skin conditioning agent is present in the range of from about 0.01 to about 5% by wt. of the total composition.

15. The liquid cleansing and moisturizing composition of claim 14 wherein the cationic skin conditioning agent is present in the range of from about 0.1 to about 1% by wt. of the total composition.

16. The composition of claim 13 wherein the cationic skin conditioning agent is selected from cationic polysaccharides, cationic copolymers of saccharides and synthetic cationic monomers, synthetic cationic polymers, polymeric quaternary ammonium salts of hydroxyethylcellulose, cationic proteins, and salts and derivatives thereof.

17. The composition of claim 1, wherein the anionic surfactant is selected from alkyl ether sulfate, alkyl sulfate, acyl isethionate, mono-and di-alkyl phosphate, and blends thereof.

18. The composition of claim 3, wherein the amphoteric/zwitterionic surfactant is selected from cocoamidopropyl betaine, sodium lauroamphoacetate, sodium cocoamphoacetate, and blends thereof.

19. The composition of claim 6, wherein the lamellar structurant is selected from isostearic acid, lauric acid, oleic acid, palm kernel acid, coconut acid, and blends thereof

20. The composition of claim 1, wherein the initial viscosity is greater than about 40,000 cps measured at 10 Pa at 25 C.

21. The composition of claim 20 wherein initial viscosity is in the range of about 40,000 to about 2,000,000 cPs measured at 10 Pa at 25 C.

22. A composition according to claim 1 further comprising a solubilizing agent.

23. The composition of claim 18 wherein the solubilizing agent is selected from isopropyl palmitate and isopropyl myristate.

24. The composition of claim 7 further comprising:
about 0.1% to about 5% by wt. of the neat cleansing lotion of a lamellar stabilizing material consisting of a polymeric hydrophilic emulsifier modified at one or both ends with hydrophobic polyhydroxy fatty acid ester chain.

25. The composition of claim 20 wherein the emulsifier is dipolyhydroxystearate.

26. The composition of claim 20 wherein the emulsifier has a polyalkylene glycol backbone chain of general formula:
H (0 (CH2) a) nOH wherein a is 2 to 4 and n is 2 to 60 having 1 to 50 C8 to C24 fatty acid group or groups attached to one or both sides of the backbone.

27. The composition of claim 22 wherein the fatty acid group or groups attached to backbone chain is selected from hydroxystearic acid, palmitic acid, and blends thereof.

28. The composition of claim 1 wherein the least one volatile foaming agent is a hydrocarbon or a mixture thereof.

29. A liquid cleansing and moisturizing composition and dispensing system comprising:
(a) from about 80 to about 97% by wt. of a neat cleansing lotion having
about 0.5 to about 65% (broad); about 1 to about 25 (narrow) by wt. of the total composition of at least one non-soap anionic or mixture of non-soap anionic surfactants;
about 35 to about 90% by wt. of the total composition of water;
(b) from about 3 to about 20% by wt. of the total composition of a volatile foaming agent;
wherein the neat cleansing lotion is a lamellar structured shear thinning composition at 25 C; and wherein the cleansing composition is contained in an aerosol pressurized container having a barrier separating the cleansing composition from an aerosol propellant.

30. The composition of claim 29 wherein the at least one non-soap anionic or mixture of non-soap anionic surfactants is in the concentration range of about 1 to about 25% by wt. of the total composition.

31. The composition and dispensing system of claim 29 wherein the pressurized barrier container is an aerosol piston can.

32. The composition and dispensing system of claim 29 wherein the composition has a dynamic density of greater that about 0.2 g/mL as measured 30 seconds after dispensing at 25 C and 1 atm pressure.

33. The composition and dispensing system of claim 32 wherein the composition has a dynamic density of greater that about 0.4 g/mL as measured 30 seconds after dispensing at 25 C and 1 atm pressure.

34. A liquid cleansing and moisturizing composition and dispensing system comprising:
(a) from about 80 to about 97% by wt. of a neat cleansing lotion having
about 0.5 to about 65% by wt. of the total composition of at least one non-soap anionic or mixture of non-soap anionic surfactants;
less than about 4% of a soap;
about 35 to about 90% by wt. of the total composition of water;
(b) from about 3 to about 20% by wt. of the total composition of a volatile foaming agent; and
wherein the neat cleansing lotion is a lamellar structured shear thinning composition at 25 C; and wherein the cleansing composition is contained in an aerosol pressurized piston container having an aerosol propellant.

35. The composition of claim 34 wherein the at least one non-soap anionic or mixture of non-soap anionic surfactants is in the concentration range of about 1 to about 25% by wt. of the total composition.

36. The composition of claim 34 wherein the soap is less than about 1% by wt. of the total composition.
